(19) United States
(12) Patent Application Publication
(10) Pub. No.: US 2010/0274094 A1
(43) Pub. Date: Oct. 28, 2010

(54) Tissue Retraction Apparatus

(75) Inventors: Mahmoud F. Abdelgany, Rockaway, NJ (US); Young Hoon Oh, Montville, NJ (US)

Correspondence Address: Rahman LLC, 10025 Governor Warfield Parkway, Suite 110, Columbia, MD, 21044, US

(73) Assignee: CUSTOM SPINE, INC., Parsippany, NJ (US)

(21) Appl. No.: 12/428,641

(22) Filed: Apr. 23, 2009

(51) Int. Cl. A61B 1/32

(52) U.S. Cl. 600/207

(57) ABSTRACT

A tissue retraction apparatus comprising a first body including a first body lower surface with a plurality of tracks embedded therein; a second body coupled to the first body that allows rotational movement of the first body relative to the second body, the second body including a second body upper surface comprising a plurality of tracks embedded therein; and a plurality of dilation components axially spaced around a dynamic opening, each dilation component comprising an arm including a top and a bottom, where the arm is coupled to an arm track of the second body and allows translational movement of the arm along the arm track; a pin fixedly coupled to the top of the arm, wherein the pin is coupled to a track of the first body and allows translational movement of the pin along the track; and a leg fixedly coupled to the bottom of the arm.

### BACKGROUND

1. Technical Field

The embodiments herein generally relate to surgical instruments, and, more particularly, to mechanical assistance of tissue retraction.

2. Description of the Related Art

Traditional surgical procedures for pathologies located within the body can cause significant trauma to the intervening tissues. These procedures often require a long incision, extensive muscle stripping, prolonged retraction of tissues, denervation and devascularization of tissue. These procedures can require operating room time of several hours and several weeks of post-operative recovery time due to the destruction of tissue during the surgical procedure. In some cases, these invasive procedures lead to permanent scarring and pain that can be more severe than the pain leading to the surgical intervention.

The development of percutaneous procedures has yielded a major improvement in reducing recovery time and post-operative pain because minimal dissection of tissue, such as muscle tissue, is required. For example, minimally invasive surgical techniques are desirable for spinal and neurosurgical applications because of the need for access to locations within the body and the danger of damage to vital intervening tissues. While developments in minimally invasive surgery are steps in the right direction, there remains a need for further development in minimally invasive surgical instruments and methods. For example, conventional systems which employ minimally invasive surgical instruments are restricted to translational movement or, if a rotational movement is employed, use relatively small rotational forces for tissue retraction. In both instances, significant force may be necessary to effectively retract tissue during a surgical procedure.

### SUMMARY

In view of the foregoing, an embodiment herein provides a tissue retraction apparatus comprising a first body component including a first body lower surface with a plurality of first hinge tracks embedded therein; a second body component coupled to the first body component that allows rotational movement of the first body relative to the second body component, the second body component including a second body upper surface comprising a plurality of arm tracks embedded therein; and a plurality of dilation components axially spaced around a dynamic opening, each dilation component comprising: an arm portion including a first end and a second end, where the arm portion is coupled to an arm track of the second body component and allows translational movement of the arm portion along the arm track; a hinge pin fixedly coupled to the first end of the arm portion, wherein the hinge pin is coupled to a hinge track of the first body component and allows translational movement of the hinge pin along the hinge track; and a leg portion fixedly coupled to the second end of the arm portion.

Two dilation components may form the dynamic opening. Additionally, four dilation components form the dynamic opening. Eight dilation components may also form the dynamic opening. In addition, the hinge tracks may be offset relative to each other. Alternatively, the hinge tracks are slanted relative to each other. The hinge tracks may also be curved. Moreover, the leg portion may include at least one of a convexed end and a concaved end. The first body component may also include a flanged outer periphery.

Additionally, a third body component may be positioned between the first body component and the second body component, wherein the third body component comprises a plurality of second hinge tracks embedded therein. Moreover, according to further embodiment, a first set of the dilation components are coupled to the first hinge tracks and a second set of the dilation components are coupled to the second hinge tracks, and a first rotational movement applied to the first body component is converted to a first translational movement of the first set of dilation components and a second rotational movement applied to the third body component is converted to a second translational movement of the second set of dilation components.

An embodiment herein provides a tissue retraction apparatus comprising a first body component including a first body lower surface with a plurality of first hinge tracks embedded therein; a second body component coupled to the first body component that allows rotational movement of the first body relative to the second body component, the second body component including a second body upper surface comprising a plurality of arm tracks embedded therein; a third body component positioned between the first body component and the second body component, wherein the third body component comprises a plurality of second hinge tracks embedded therein; and a plurality of dilation components axially spaced around a dynamic opening, wherein each dilation component comprises: an arm portion including a first end and a second end, wherein the arm portion is coupled to an arm track of the second body component and allows translational movement of the arm portion along the arm track; a hinge pin fixedly coupled to the first end of the arm potion, wherein the hinge pin is coupled to a hinge track of the first body component and allows translational movement of the hinge pin along the hinge track; and a leg portion fixedly coupled to the second end of the arm portion.

In addition, a first set of the dilation components may be coupled to the first hinge tracks and a second set of the dilation components may be coupled to the second hinge tracks, and when a first rotational movement applied to the first body component, it is converted to a first translational movement of the first set of dilation components and when a second rotational movement applied to the third body component, it is converted to a second translational movement of the second set of dilation components. Moreover, the hinge tracks may be offset relative to each other. The hinge tracks may also be slanted relative to each other. Furthermore, the hinge tracks may also be curved. Additionally, the leg portion may include at least one of a convexed end and a concaved end. The first body component may also include a flanged outer periphery.

An embodiment herein provides a tissue retraction apparatus comprising a first body component including a first body lower surface with a plurality of first hinge tracks embedded therein; a second body component coupled to the first body component that allows rotational movement of the first body component relative to the second body component, the second body component including a second body upper surface with a plurality of arm tracks embedded therein; a third body component positioned between the first body component and the second body component, the third body component comprising at least one second hinge track embedded therein; a third fourth component situated between the first body component and the second body component and adjacent to the third body component, the fourth body component comprising at least one third hinge track embedded therein; and a plurality of dilation components axially spaced around a dynamic opening, wherein each dilation component comprises: an arm portion including a first end and a second end, where the arm portion is coupled to an arm track of the second body component and allows translational movement of the arm portion along the arm track; a hinge pin fixedly coupled to the first end of the arm portion, wherein the hinge pin is coupled to a hinge track of the first body component and allows translational movement of the hinge pin along the hinge track; and a leg portion fixedly coupled to the second end of the arm portion.

Such an embodiment may have a first set of the dilation components which are coupled to the first hinge tracks, a second set of the dilation components which are coupled to the second hinge tracks, and a third set of dilation components which are coupled to the third hinge tracks, and a first rotational movement applied to the first body component that is converted to a first translational movement of the first set of dilation components, a second rotational movement applied to the third body component that is converted to a second translational movement of the second set of dilation components, and a third rotational movement applied to the fourth body component that is converted to a third translational movement of the third set of dilation components.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The embodiments herein will be better understood from the following detailed description with reference to the drawings, in which:

FIG. 1 illustrates a schematic diagram of a tissue retraction device with two axially spaced dilation components, shown in an unexpanded configuration, according to one embodiment described herein;

FIG. 2 illustrates a schematic diagram of a tissue retraction device with two axially spaced dilation components, shown in an expanded configuration, according to one embodiment described herein;

FIG. 3 illustrates a schematic diagram of a tissue retraction device with four axially spaced dilation components, shown in an unexpanded configuration, according to one embodiment described herein;

FIG. 4 illustrates a schematic diagram of a tissue retraction device with four axially spaced dilation components, shown in an expanded configuration, according to one embodiment described herein;

FIGS. 5A-5C illustrate a tissue retraction device with eight axially spaced dilation components in an unexpanded configuration in three separate orientations, according to one embodiment described herein;

FIGS. 6A-6C illustrate a tissue retraction device with eight axially spaced dilation components in an expanded configuration in three separate orientations, according to one embodiment described herein;

FIGS. 7A-7D illustrate a dilation component in four separate orientations, according to one embodiment described herein;

FIGS. 8A-8D illustrate an arm portion of a dilation component, in four separate orientations, according to one embodiment described herein;

FIGS. 9A-9D illustrate a leg portion of a dilation component, in four separate orientations, according to one embodiment described herein;

FIGS. 10A-10C illustrate a bottom component, in three separate orientations, according to one embodiment described herein;

FIGS. 11A-11C illustrate a top component, in three separate orientations, according to one embodiment described herein;

FIG. 12 is a schematic diagram illustrating an alternative embodiment of the tissue retraction device, in an unexpanded configuration, according to one embodiment described herein;

FIG. 13 is a schematic diagram illustrating an alternative embodiment of the tissue retraction device, in an expanded configuration, according to one embodiment described herein;

FIG. 14 is a schematic diagram illustrating another alternative embodiment of the tissue retraction device, in an unexpanded configuration, according to one embodiment described herein; and

FIG. 15 is a schematic diagram illustrating another alternative embodiment of the tissue retraction device, in an expanded configuration according, to one embodiment described herein.

### DETAILED DESCRIPTION OF PREFERRED EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

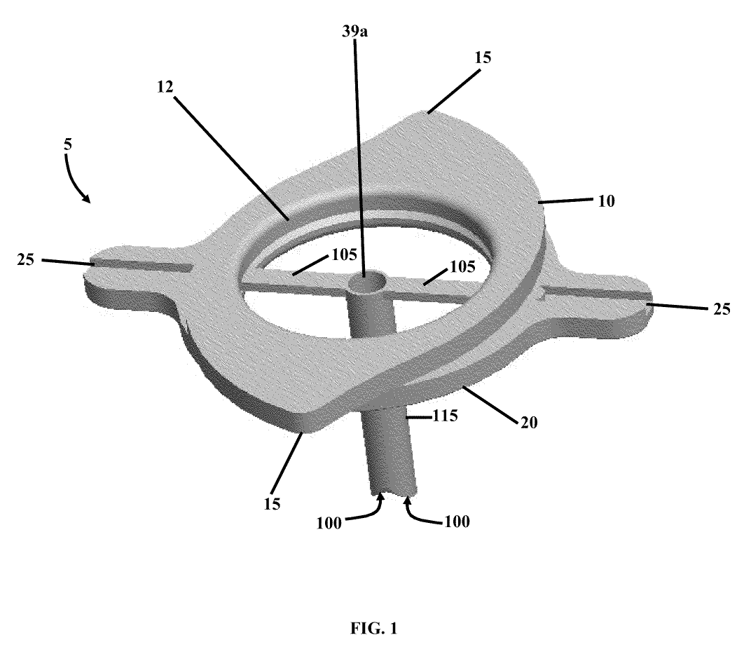

As previously mentioned, there remains a need to retract the tissue while requiring minimal force from the user. The embodiments herein achieve this by providing a large diameter of rotation such that the rotational movement is converted into translational movement to retract the tissue, and thereby needing less force from the user. In addition, the embodiments described herein provide both translating and rotating movement to increase the dynamic opening and tissue translation in different directions. Referring now to the drawings, and more particularly to FIGS. 1 through 15, where similar reference characters denote corresponding features consistently throughout the figures, there are shown preferred embodiments.

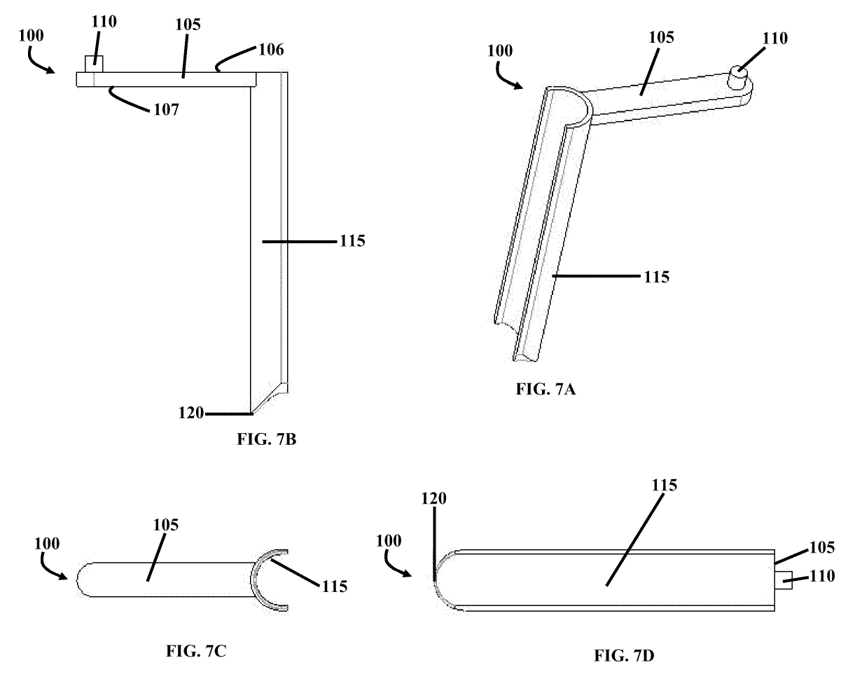

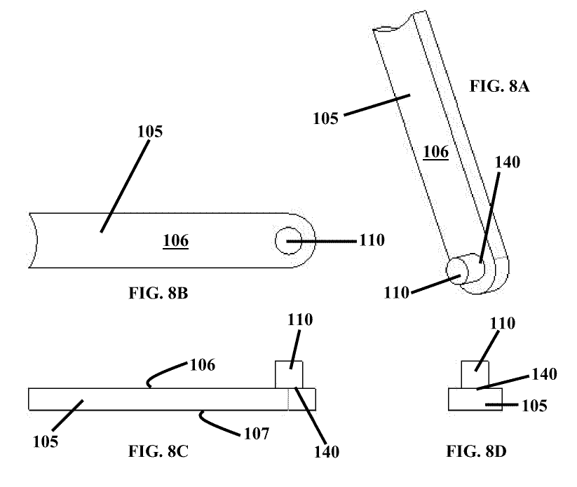

FIG. 1, with reference to FIGS. 7A through 8D, is a top perspective view of a tissue retraction device 5 with two axially spaced dilation components 100, in an unexpanded configuration, according to one embodiment described herein. Tissue retraction device 5 comprises a top component (or first body) 10, a bottom component (or a second body) 20, and a plurality of dilation components 100 comprising arm portions 105 and leg portions 115. Both top component 10 and bottom component 20 together form working channel 12, which is shown as a fixed opening in FIG. 1. Additionally, top component 10 optionally has flanged components 15 attached thereto. Top component 10 also has a plurality of hinge tracks etched in a lower surface thereof (not shown in FIG. 1, but illustrated in further detail below). Bottom component 20 has a plurality of arm tracks 25. As shown in FIG. 1, the number of arm tracks 25 corresponds to the number of arm portions 105 coupled to each dilation component 100 as described in further detail below. As further described below with reference to FIGS. 7A through 8D, each dilation component 100 includes an arm portion 105, a leg portion 115, and a hinge pin 110. The arm tracks 25 are dimensioned and configured to receive the hinge pin 110 of each dilation component 100. Joined together, the leg portions 115 of the dilation components 100 form dynamic opening 39a in the unexpanded configuration of the device 5 of FIG. 1. As a result of dynamic opening 39a, tissue retraction device 5 in an unexpanded configuration can be easily inserted into a small incision.

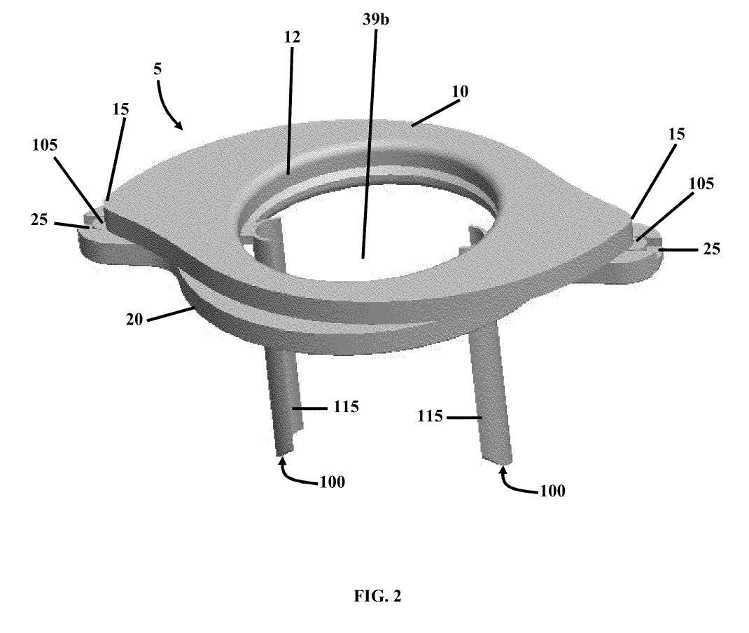

FIG. 2, with reference to FIGS. 1 and 7A through 8D, is a top perspective view of the tissue retraction device 5 of FIG. 1 with two axially spaced dilation components 100, in an expanded configuration, according to one embodiment described herein. In the expanded configuration shown in FIG. 2, tissue retraction device 5 has increased the axial spacing of the individual dilation components 100 by rotating the top component 10 (optionally via flanged components 15) relative to bottom component 20. The rotational movement of top component 10 allows the arm portions 105 of the dilation components 100 to perform translational movement along arm tracks 25. Converting the rotational movement of top component 10 into the translational movement of the dilation components 100 is accomplished via hinge pins 110, which are fixedly coupled to the arm portions 105 and coupled to hinge tracks (not shown in FIG. 2) on the lower surface of top component 10, as described in more detail below. As a consequence of the translational movement incident to the rotational movement applied to top component 10, the leg portions 115 of the dilation components 100 form dynamic opening 39b in device 5. In the expanded configuration, tissue retraction device 5 provides a greater working area than otherwise available in the unexpanded configuration described in FIG. 1.

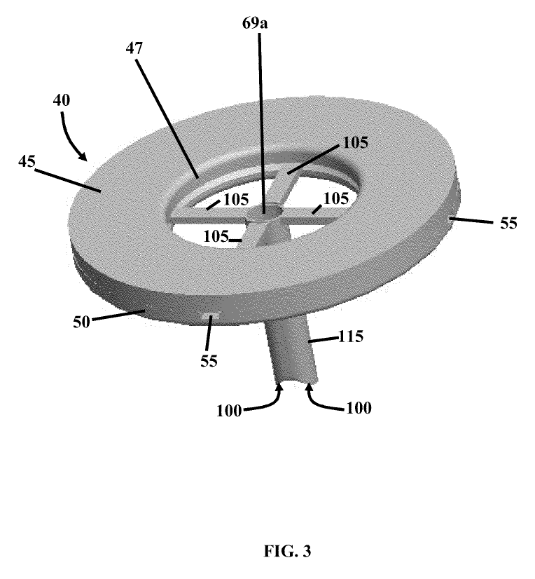

FIG. 3, with reference to FIGS. 7A through 8D, is a schematic diagram illustrating a tissue retraction device 40 with four axially spaced dilation components 100, in an unexpanded configuration, according to one embodiment described herein. Tissue retraction device 40 comprises a top component 45, a bottom component 50, and a plurality of dilation components 100 comprising arm portions 105. Both top component 45 and bottom component 50 together form working channel 47, which is shown as a fixed opening in FIG. 3. In this embodiment, top component 45 and bottom component 50 are circular in shape. While not shown in FIG. 3, top component 45 has a plurality of hinge tracks etched within a lower surface thereon. Similarly, bottom component 50 has a plurality of arm tracks 55 etched within an upper surface thereon. As with the device 5 shown in FIGS. 1 and 2, the number of arm tracks 55 corresponds to the number of arm portions 105 coupled to each dilation component 100 as described in further detail below. Furthermore, as with the device 5 shown in FIGS. 1 and 2 and with reference to FIGS. 7A through 8D, each dilation component 100 in FIG. 3 includes an arm portion 105 positioned between a leg portion 115 and a hinge pin 110. Joined together, leg portions 115 of the dilation components 100 form a dynamic opening 69a in the unexpanded configuration of device 40 in FIG. 3. As a result of dynamic opening 69a, tissue retraction device 40 in an unexpanded configuration can be easily inserted into a small incision.

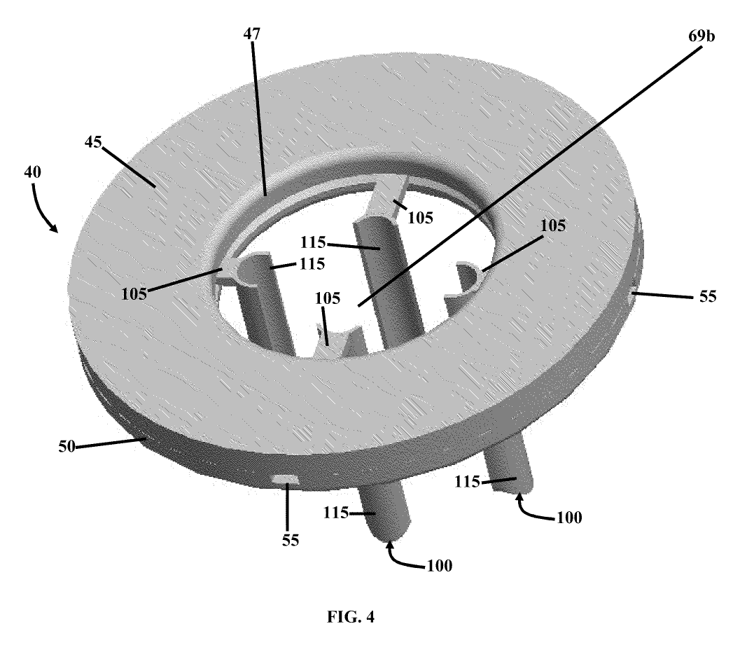

FIG. 4, with reference to FIGS. 3 and 7A through 8D is a top perspective view of the tissue retraction device 40 of FIG. 3 with four axially spaced dilation components 100, in an expanded configuration, according to one embodiment described herein. In the expanded configuration shown in FIG. 4, tissue retraction device 40 has increased the axial spacing of the individual dilation components 100 by rotating the top component 45 relative to bottom component 50. The rotational movement of top component 45 allows the arm portions 105 of dilation components 100 to perform translational movement along arm tracks 55. Converting the rotational movement of top component 45 into the translational movement of dilation components 100 is accomplished via hinge pins 110, which are fixedly coupled to the arm portions 105 and coupled to hinge tracks (not shown in FIG. 4) on the lower surface of top component 45. As a consequence of the translational movement incident to the rotational movement applied to top component 45, leg portions 115 of dilation components 100 form dynamic opening 69b in the expanded configuration of device 40 of FIG. 4. In the expanded configuration, tissue retraction device 40 provides a greater working area than otherwise available in the unexpanded configuration described in FIG. 3.

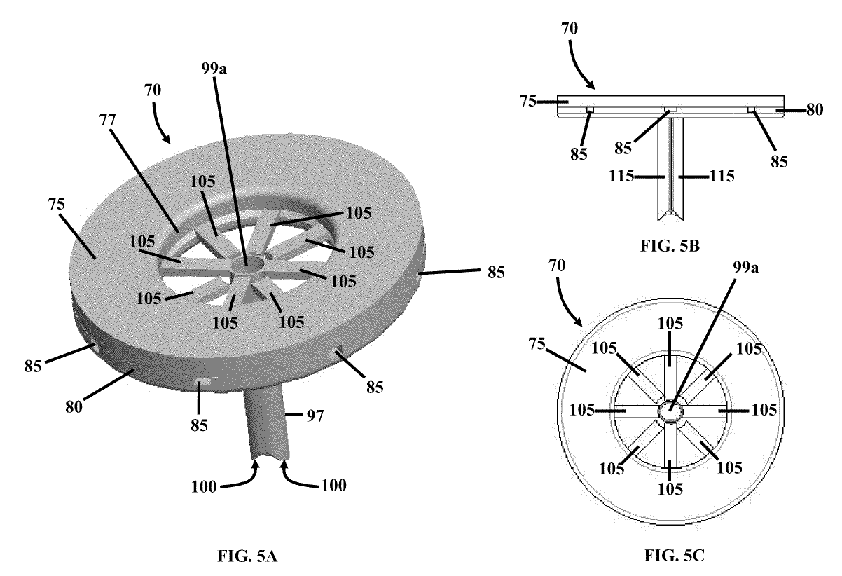

FIGS. 5A through 5C, with reference to FIGS. 7A through 8D, are schematic diagrams illustrating various views of a tissue retraction device 70 with eight axially spaced dilation components 100, in an unexpanded configuration, according to one embodiment described herein. In FIG. 5A, a top perspective view of tissue retraction device 70 is illustrated comprising a top component 75, a bottom component 80, and a plurality of dilation components 100 comprising arm portions 105. Both top component 75 and bottom component 80 together form working channel 77, which is shown as a fixed opening in FIG. 5A. While not shown in FIG. 5A, top component 75 additionally has a plurality of hinge tracks etched within a lower surface thereon. Bottom component 80 has a plurality of arm tracks 85 etched in the upper surface thereon. In the embodiment shown in FIG. 5A, the number of arm tracks 85 corresponds to the number of arm portions 105 coupled to each dilation component 100. In accordance with FIGS. 7A through 8D, each dilation component 100 includes an arm portion 105 situated between a leg portion 115 and a hinge pin 110. Joined together, leg portions 115 of dilation components 100 form dynamic opening 99a of the unexpanded configuration of device 70 shown in FIGS. 5A through 5C. As a result of dynamic opening 99a, tissue retraction device 70 in an unexpanded configuration can be easily inserted into a small incision.

FIG. 5B shows a side elevation view of tissue retraction device 70 of FIG. 5A. As shown, top component 75 is coupled to bottom component 80. In addition, FIG. 5B illustrates bottom component 80 having a number of arm tracks 85 etched in its upper surface. Also shown are two leg portions 115 of dilation components 100 in the unexpanded configuration. FIG. 5C shows a plan view of tissue retraction device 70 of FIG. 5A. The arm portions 105 of the eight axially spaced dilation components 100 are shown in the unexpanded configuration to form the small opening 99a. In addition, top component 75 is shown surrounding axially arm portions 105.

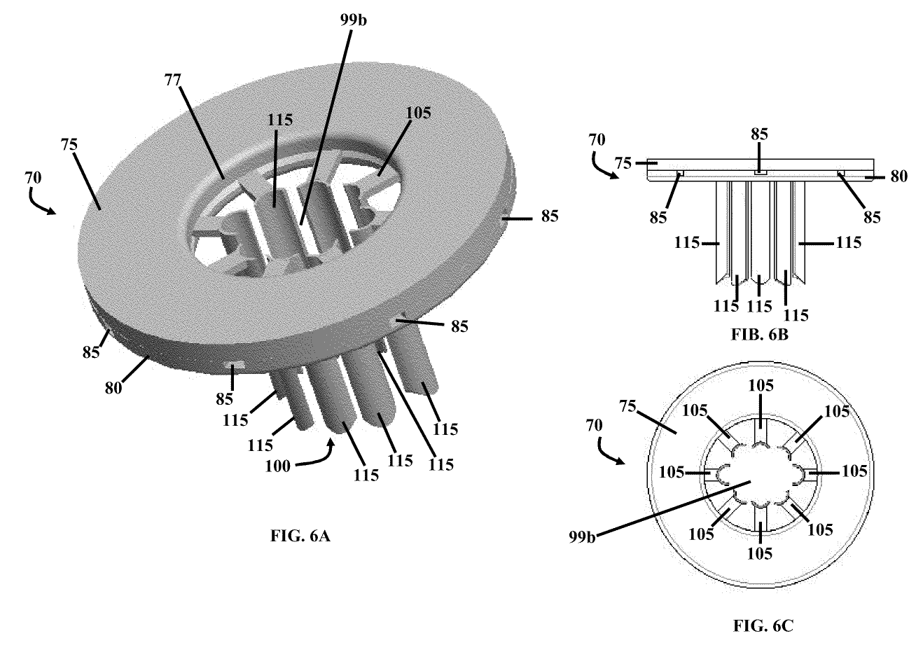

FIGS. 6A through 6C, with reference to FIGS. 5A through 5C and 7A through 8D, are schematic diagrams illustrating various views of a tissue retraction device 70 with eight axially spaced dilation components 100, in an expanded configuration, according to one embodiment described herein. In FIG. 6A, the expanded configuration tissue retraction device 70 is shown in a front perspective view. Due to the rotation of top component 75 relative to the bottom component 80, dilation components 100 have formed a dynamic opening 99b, which has a greater working area then what was shown in the device 70 in the unexpanded configuration of FIG. 5A. The rotational movement of top component 75 allows the arm portions 105 of dilation components 100 to perform translational movement along arm tracks 85. Converting the rotational movement of top component 75 into the translational movement of dilation components 100 is accomplished via hinge pins 110, which are attached to the arm portions 105 and coupled to hinge tracks (not shown) on the lower surface of top component 75. As a consequence of the translational movement incident to the rotational movement applied to top component 75, the leg portions 115 of the dilation components form dynamic opening 99b in the expanded configuration of device 70 shown in FIGS. 6A through 6C. In the expanded configuration, tissue retraction device 70 provides a greater working area than otherwise available in the unexpanded configuration described in FIGS. 5A through 5C.

FIG. 6B shows a side elevation view of tissue retraction device 70. As described with respect to FIG. 5B, top component 75 is shown coupled to bottom component 80 and FIG. 6B illustrates bottom component 80 having a number of arm tracks 85 etched in its upper surface. In addition, FIG. 6B shows five of the eight leg portions 115 of the dilation components 100 in the expanded configuration. FIG. 6C shows a plan view of tissue retraction device 70. As illustrated, the arm portions 105 of the eight axially spaced dilation components 100 are shown in the expanded configuration to form the dynamic opening 99b.

FIGS. 7A through 7D are schematic diagrams illustrating various views of a dilation component 100, according to one embodiment described herein. FIG. 7A is a side perspective view of dilation component 100. As shown, the dilation component 100 comprises arm portion 105, hinge pin 110, and a leg portion 115. In FIG. 7A, hinge pin 110 is fixedly coupled to a first end of arm portion 105. Additionally, leg portion 115 is fixedly coupled to a second end of arm portion 105. As a result of the fixed couplings, the various parts of dilation component 100 move in unison. FIG. 7B illustrates a side elevation view of dilation component 100. In addition to showing the features of dilation component 100 described above, FIG. 7B also illustrates an optional convex end 120 of leg portion 115. Additionally, while not shown in FIG. 7B, leg portion 115 may have an optional concave end. FIG. 7C is a plan view of dilation component 100. In addition to showing the fixed coupling of arm portion 105 to leg portion 115, FIG. 7C also illustrates leg portion 115 as optionally being convexed throughout its entire length. FIG. 7D is a back elevation view of dilation component 100 to show hinge pin 110, leg portion 115, and the convexed end 120 of leg portion 115. Arm portion 105 is fixedly coupled to hinge pin 110 at one end and fixedly coupled to leg portion 115 at the other end. The hinge pin 110 may be configured on either the upper surface 106 of the arm portion 105 or the lower surface 107 of the arm portion 105.

FIGS. 8A through 8D are schematic diagrams illustrating various views of one embodiment of the arm portion 105 of the dilation component 100 of FIGS. 7A through 7D. FIG. 8A is a side perspective view of arm portion 105. As shown, the arm portion 105 connects to hinge pin 110 via joint 140. FIG. 8B is a plan view of arm portion 105 to further illustrate the hinge pin 110 extending outwardly from the upper surface 106 of the arm portion 105. FIG. 8C is a side elevation view of arm portion 105 illustrating the relative thicknesses of hinge pin 110 and arm portion 105. FIG. 8D is a front elevation view of arm portion 105 and hinge pin 110.

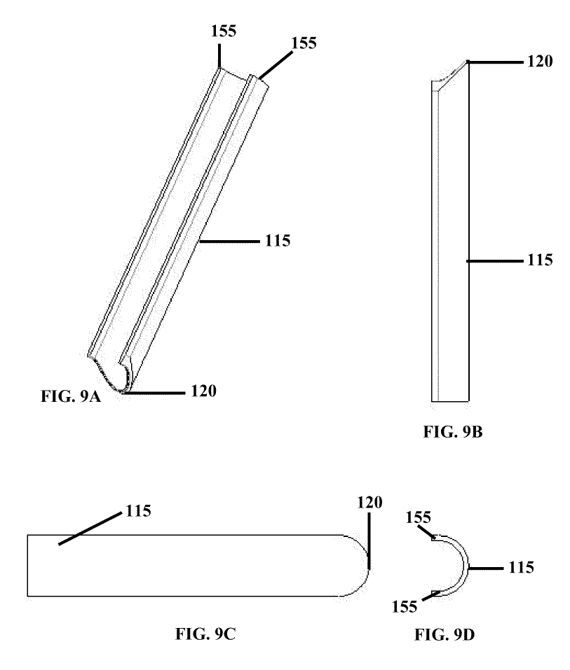

FIGS. 9A through 9D are schematic diagrams illustrating various views of one embodiment of a leg portion 115 of the dilation component 100 of FIGS. 7A through 7D. FIG. 9A is a side perspective view of leg portion 115 with edges 155 and convexed end 120. Optionally, edges 155 are notched so each can accommodate an adjacent leg portion 115 of another dilation component 100. FIG. 9B is a side elevation view of leg portion 115 to further illustrate the convexed end 120. FIG. 9C is a plan view of leg portion 115 that also shows the convexed end 160. FIG. 9D is a top view of leg portion 115 that shows edges 155.

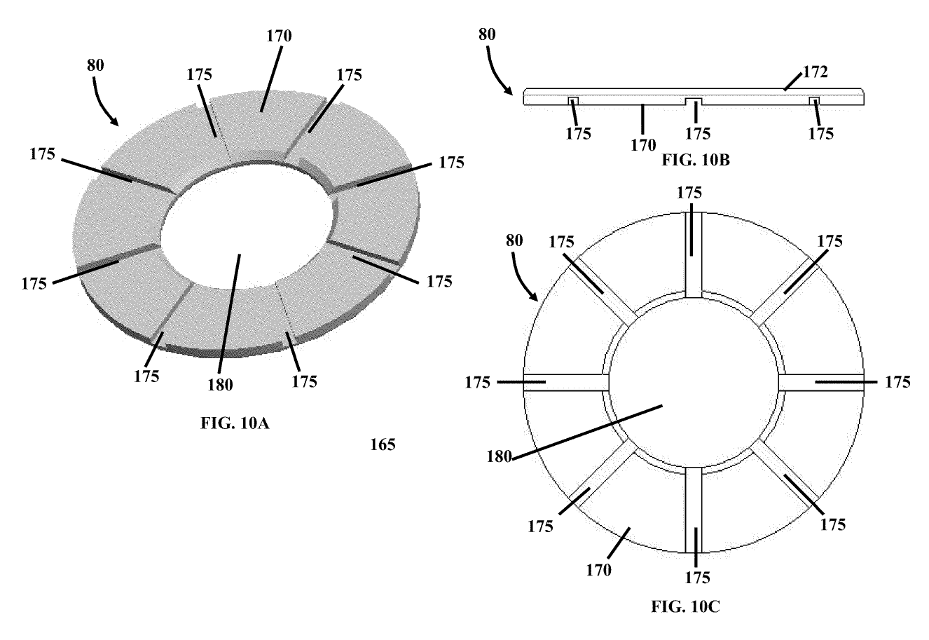

FIGS. 10A through 10C, with reference to FIGS. 5A through 8D, are schematic diagrams illustrating various views of one embodiment of a bottom component 80 of the tissue retraction device 70 of FIGS. 5A through 6C. FIG. 10A is a top perspective view of bottom component 80. As shown, bottom component 80 includes an upper surface 170, a number of etched arm tracks 175 and a working channel 180. In the embodiment shown, each arm track 175 accommodates a single arm portion 105 of a dilation component 100. In addition, each arm portion 105 is loosely coupled to an arm track 175 to allow translational movement of the arm portion 105 of the dilation component 100 along the arm track 175. In addition, working channel 180 is shown as a fixed opening in FIG. 10A.

FIG. 10B illustrates an inverted side elevation view of bottom component 80. In particular, FIG. 10B shows upper surface 170, lower surface 172, and arm tracks 175 etched in upper surface 170. FIG. 10C is a plan view of bottom component 80 and illustrates upper surface 170, arm tracks 175, and working channel 180.

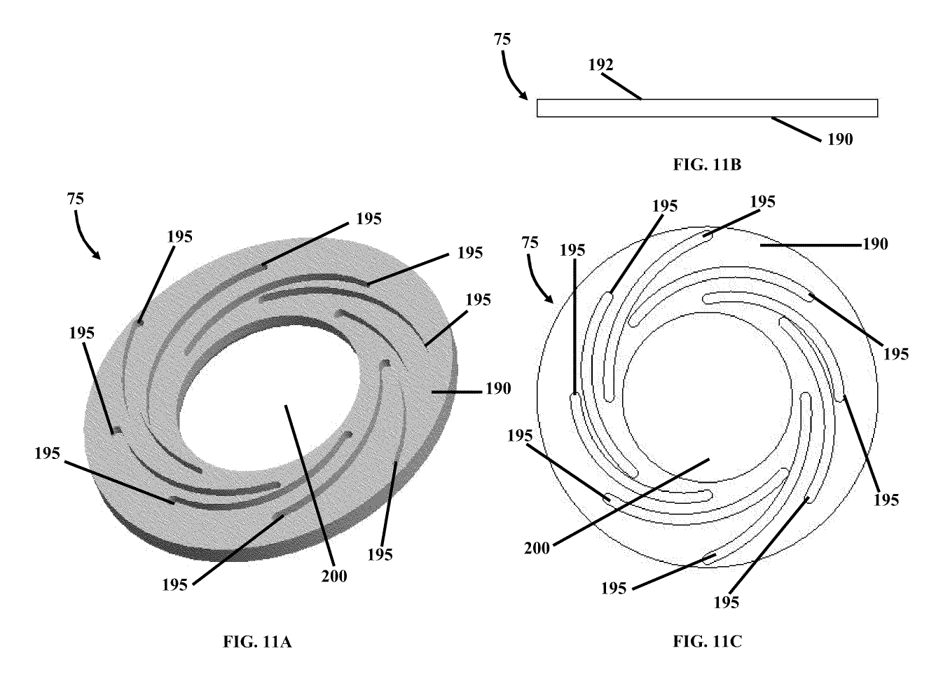

FIGS. 11A through 11C, with reference to FIGS. 5A through 8D, are schematic diagrams illustrating various views of one embodiment of a top component 75 of the tissue retraction device 70 of FIGS. 5A through 6C. FIG. 11A is a bottom perspective view of top component 75. As shown, top component 75 includes a lower surface 190, a plurality of etched hinge tracks 195 and a working channel 200. In the embodiment shown, hinge tracks 195 are curved or arced and each accommodates a single hinge pin 110 (shown in FIGS. 7A through 8D) of a dilation component 100 (as described above). In addition to being curved, other hinge track configurations are possible. For example, instead of being curved, hinge tracks 195 could be slanted or simply offset relative to each other. Within hinge tracks 195, hinge pins 110 are loosely coupled therein to convert the rotational movement of the top component 75 into translational movement of the arm portion 105 of the dilation component 100. In addition, similar to the working channel 180 illustrated in bottom component 80, working channel 200 of the top component 75 is shown as a fixed opening in FIG. 11A. FIG. 11B illustrates a side elevation view of top component 75. In particular, FIG. 11B shows lower surface 190 and upper surface 192. Moreover, FIG. 11C is a plan view of top component 75 and illustrates lower surface 190, hinge tracks 195 and working channel 200.

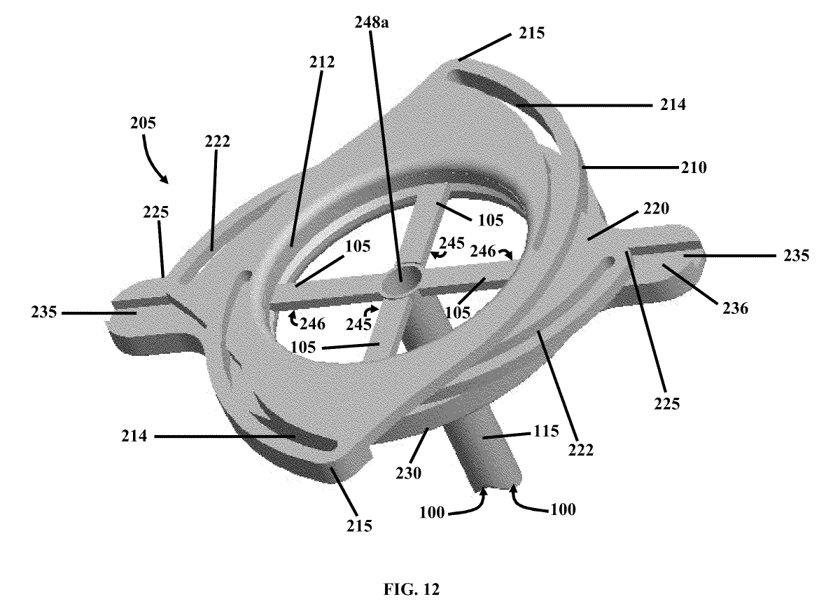

FIG. 12, with reference to FIGS. 7A through 8D, is a top perspective view of an alternative embodiment of a tissue retraction device 205, in an unexpanded configuration according to one described herein. Tissue retraction device 205 comprises a top component (or first body) 210, a bottom component (or a second body) 230, an intermediate component 220 (or third body) situated between top component 210 and bottom component 230 and a plurality of dilation components 100. Both the top component 210 and the intermediate component 220 allow rotational movement relative to each other and relative to bottom component 230. Moreover, top component 210, intermediate component 220, and bottom component 230 together form working channel 212, which is shown as a fixed opening in FIG. 12. Both top component 210 and intermediate component 220 optionally have flanged components 215, 225 attached thereto, respectively. Also shown are a plurality of hinge tracks 214, 222 embedded through the top component 210 and the intermediate component 220, respectively. Bottom component 230 has a plurality of arm tracks 235 etched on an upper surface 236 thereon. As discussed above, each dilation component 100 includes an arm portion 105 positioned between a hinge pin 110 and a leg portion 115.

In FIG. 12, dilation components 100 may be partition into a first set of dilation components 245 coupled to top component 210 and a second set of dilation components 246 coupled to intermediate component 220. Movement of the first set of dilation components 245 is severable from movement of the second set of dilation components 246 because the first set of dilation components 245 is coupled to the hinge tracks 214 of top component 210 and the second set of dilation components 246 is coupled to the hinge tracks 222 of the intermediate component. Thus, translational movement of the first set of dilation components 245 is incident to the rotation of top component 210 and translational movement of the second set of dilation components 246 is incident to the rotation of intermediate component 220.

Joined together, leg portions 115 of the first set of dilation components 245 and the second set of dilation components 246 form dynamic opening 248a in the unexpanded configuration of the device 205 shown in FIG. 12. As a result of dynamic opening 248a, tissue retraction device 205 in an unexpanded configuration can be easily inserted into a small incision.

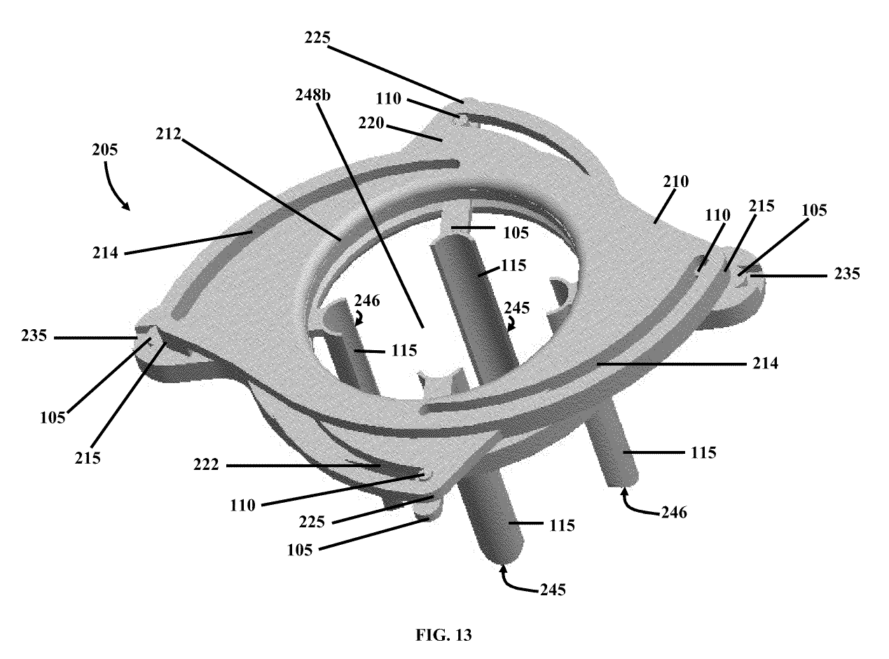

FIG. 13, with reference to FIGS. 7A through 8D and FIG. 12, is a top perspective view of the alternative embodiment of the tissue retraction device 205 of FIG. 12, in an expanded configuration according to one described herein. In the expanded configuration, tissue retraction device 205 has increased the axial spacing of the individual dilation components 100 by rotating at least one of top component 210 (optionally via flanged components 215 and intermediate component 220 (optionally via flanged components 225) relative each other and to bottom component 230. The rotational movement of top component 210 allows the arm portions 105 of the dilation components 100 to perform translational movement along arm the tracks 235. Similarly, the rotational movement of intermediate component 220 allows the arm portions 105 of dilation components 100 to perform translational movement along the arm tracks (not shown). Converting the rotational movement of at least one of top component 210 and intermediate component 220 into the translational movement of the dilation components 100 is accomplished via hinge pins 110, which are fixedly coupled to the arm portions 105 and coupled to hinge tracks 214, 22 embedded in at least one of top component 210 and intermediate component 220, respectively. As a consequence of the translational movement incident to the rotational movement applied to at least one of top component 210 and intermediate component 220, the leg portions 115 of the dilation components 100 form dynamic opening 248b in the expanded configuration of device 205.

While dynamic opening 248b is relatively uniform in FIG. 13, those skilled in art would understand that other configurations are possible. For example, if the first set of dilation components 245 are subject to greater translational movement compared to the second set of dilation components 246, then the dynamic opening 248b would be roughly elliptical in shape. Thus, in the expanded configuration of FIG. 13, tissue retraction device 205 provides a greater working area than otherwise available in the unexpanded configuration of device 205 shown in FIG. 12.

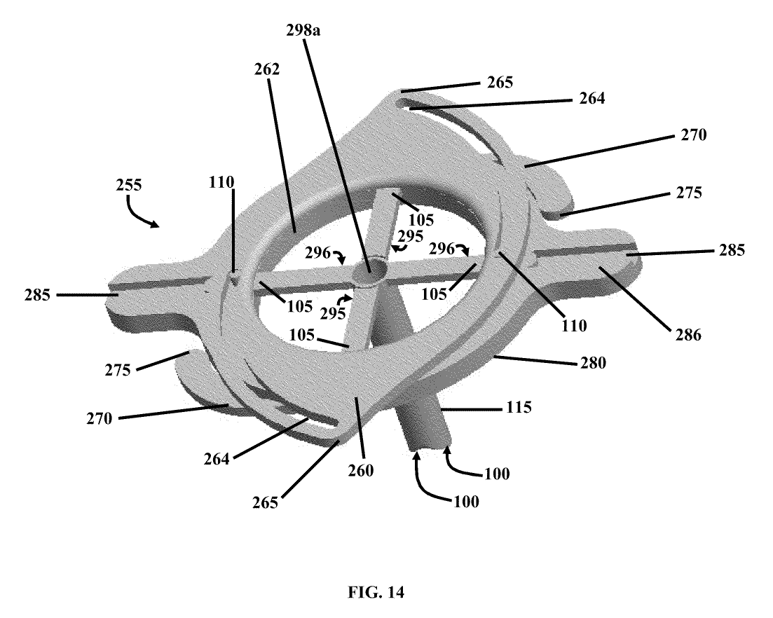

FIG. 14, with reference to FIGS. 7A through 8D, is a top perspective view of another alternative embodiment of a tissue retraction device 255, in an unexpanded configuration according to one described herein. Tissue retraction device 255 includes a top component (or first body) 260, a bottom component (or a second body), 280, intermediate component 270 situated between top component 260 and bottom component 280, and a plurality of dilation components 100. Both the top component 260 and the intermediate components 270 allow rotational movement relative to each other and relative to bottom component 280. Moreover, top component 260, intermediate component 270, and bottom component 280 together form working channel 262, which is shown as a fixed opening in FIG. 14. Additionally, both top component 260 and intermediate components 270 optionally have flanged components 265, 275 attached thereto, respectively. A plurality of hinge tracks 264 are also embedded through the top component 260 and hinge tracks (not shown in FIG. 14) are also embedded through the intermediate components 270. Bottom component 280 has a plurality of arm tracks 285 etched on an upper surface 286 thereon. As discussed above, each dilation component 100 includes an arm portion 105 situated between a hinge pin 110 and a leg portion 115.

In FIG. 14, dilation components 100 may be partitioned into a first set of dilation components 295 coupled to top component 210 and a second set of dilation components 296 coupled to intermediate components 270. Each intermediate component 270 may be rotated independently. Consequently, movement of the first set of dilation components 295 is severable from movement of each dilation component 100 in the second set of dilation components 296. Thus, translational movement of the first set of dilation components 295 is incident to the rotation of top component 210 and translational movement of the second set of dilation components 296 is incident to the rotation of intermediate components 270 (either individually or together).

Joined together, leg portions 115 of the first set of dilation components 295 and the second set of dilation components 296 form dynamic opening 298a in the unexpanded configuration of device 255 shown in FIG. 14. As a result of dynamic opening 298a, tissue retraction device 255 in an unexpanded configuration can be easily inserted into a small incision.

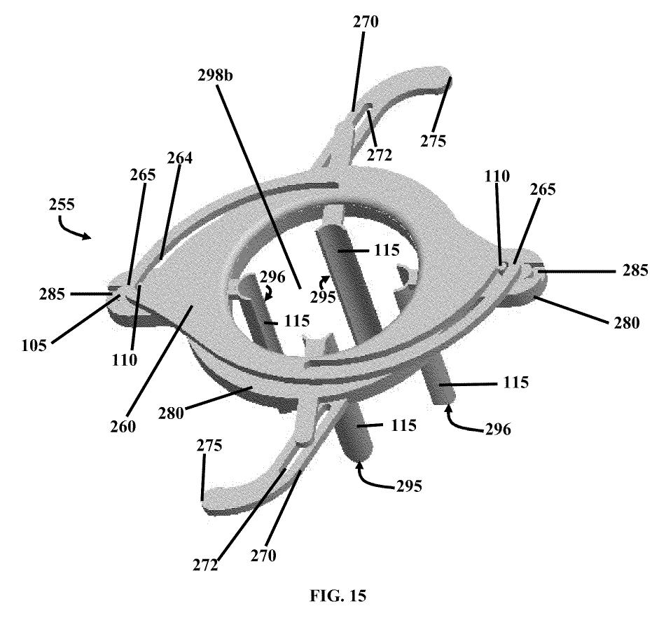

FIG. 15, with reference to FIGS. 7A through 8D, is a top perspective view of another alternative embodiment of the tissue retraction device 255, in an expanded configuration according to one described herein. In the expanded configuration, tissue retraction device 255 has increased the axial spacing of the individual dilation components 100 by rotating at least one of top component 260 (optionally via flanged components 265) and intermediate components 270 (optionally via flanged components 275) relative to each other and to bottom component 280. The rotational movement of top component 260 allows the arm portions 105 of the dilation components 100 to perform translational movement along arm tracks 285. Similarly, the rotational movement of intermediate component 270 allows the arm portions 105 of the dilation components 100 to perform translational movement along arm tracks (not shown in FIG. 15). Converting the rotational movement of at least one of top component 260 and intermediate components 270 into the translational movement of the dilation components 100 is accomplished via hinge pins 110, which are fixedly coupled to the arm portions 105 and coupled to hinge tracks 264, 272 embedded in at least one of top component 260 and intermediate components 270, respectively. As a consequence of the translational movement incident to the rotational movement applied to at least one of top component 260 and intermediate components 270, the leg portions 115 of the dilation components 100 form dynamic opening 298b in the expanded configuration of device 255 shown in FIG. 15.

While dynamic opening 298b is relatively uniform in FIG. 15, those skilled in art would understand that other configurations are possible. For example, if the first set of dilation components 295 are subject to greater translational movement compared to the second set of dilation components 296, then the dynamic opening 298b would be roughly elliptical in shape. Alternatively, dynamic opening 298b could take an amorphous shape when top component 260, intermediate component 270, and bottom component 280 are each subjected to a different degree of rotation. Thus, in the expanded configuration of FIG. 15, tissue retraction device 255 provides a greater working area than otherwise available in the unexpanded configuration described in FIG. 14.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

What is claimed is:

1. A tissue retraction apparatus comprising:
a first body component including a first body lower surface with a plurality of first hinge tracks embedded therein;
a second body component coupled to said first body component that allows rotational movement of said first body relative to said second body component, said second body component including a second body upper surface comprising a plurality of arm tracks embedded therein; and
a plurality of dilation components axially spaced around a dynamic opening, each dilation component comprising:
an arm portion including a first end and a second end, where said arm portion is coupled to an arm track of said second body component and allows translational movement of said arm portion along said arm track;
a hinge pin fixedly coupled to said first end of said arm portion, wherein said hinge pin is coupled to a hinge track of said first body component and allows translational movement of said hinge pin along said hinge track; and
a leg portion fixedly coupled to said second end of said arm portion.

2. The apparatus of claim 1, wherein two said dilation components form said dynamic opening.

3. The apparatus of claim 1, wherein four said dilation components form said dynamic opening.

4. The apparatus of claim 1, wherein eight said dilation components form said dynamic opening.

5. The apparatus of claim 1, wherein said hinge tracks are offset relative to each other.

6. The apparatus of claim 1, wherein said hinge tracks are slanted relative to each other.

7. The apparatus of claim 1, wherein said hinge tracks are curved.

8. The apparatus of claim 1, wherein said leg portion includes at least one of a convexed end and a concaved end.

9. The apparatus of claim 1, wherein said first body component includes a flanged outer periphery.

10. The apparatus of claim 1, further comprising a third body component positioned between said first body component and said second body component, wherein said third body component comprises a plurality of second hinge tracks embedded therein.

11. The apparatus of claim 10,
wherein a first set of said dilation components are coupled to said first hinge tracks and a second set of said dilation components are coupled to said second hinge tracks, and
wherein a first rotational movement applied to said first body component is converted to a first translational movement of said first set of dilation components and a second rotational movement applied to said third body component is converted to a second translational movement of said second set of dilation components.

12. A tissue retraction apparatus comprising:
a first body component including a first body lower surface with a plurality of first hinge tracks embedded therein;
a second body component coupled to said first body component that allows rotational movement of said first body relative to said second body component, said second body component including a second body upper surface comprising a plurality of arm tracks embedded therein;
a third body component positioned between said first body component and said second body component, wherein said third body component comprises a plurality of second hinge tracks embedded therein; and
a plurality of dilation components axially spaced around a dynamic opening, wherein each dilation component comprises:
an arm portion including a first end and a second end, wherein said arm portion is coupled to an arm track of said second body component and allows translational movement of said arm portion along said arm track;
a hinge pin fixedly coupled to said first end of said arm potion, wherein said hinge pin is coupled to a hinge track of said first body component and allows translational movement of said hinge pin along said hinge track; and
a leg portion fixedly coupled to said second end of said arm portion.

13. The apparatus of claim 12,
wherein a first set of said dilation components are coupled to said first hinge tracks and a second set of said dilation components are coupled to said second hinge tracks, and
wherein a first rotational movement applied to said first body component is converted to a first translational movement of said first set of dilation components and a second rotational movement applied to said third body component is converted to a second translational movement of said second set of dilation components.

14. The apparatus of claim 12, wherein said hinge tracks are offset relative to each other.

15. The apparatus of claim 12, wherein said hinge tracks are slanted relative to each other.

16. The apparatus of claim 12, wherein said hinge tracks are curved.

17. The apparatus of claim 12, wherein said leg portion includes at least one of a convexed end and a concaved end.

18. The apparatus of claim 12, wherein said first body component includes a flanged outer periphery.

19. A tissue retraction apparatus comprising:
a first body component including a first body lower surface with a plurality of first hinge tracks embedded therein;
a second body component coupled to said first body component that allows rotational movement of said first body component relative to said second body component, said second body component including a second body upper surface with a plurality of arm tracks embedded therein;
a third body component positioned between said first body component and said second body component, said third body component comprising at least one second hinge track embedded therein;
a third fourth component situated between said first body component and said second body component and adjacent to said third body component, said fourth body component comprising at least one third hinge track embedded therein; and
a plurality of dilation components axially spaced around a dynamic opening, wherein each dilation component comprises:
an arm portion including a first end and a second end, where said arm portion is coupled to an arm track of said second body component and allows translational movement of said arm portion along said arm track;
a hinge pin fixedly coupled to said first end of said arm portion, wherein said hinge pin is coupled to a hinge track of said first body component and allows translational movement of said hinge pin along said hinge track; and
a leg portion fixedly coupled to said second end of said arm portion.

20. The apparatus of claim 19,
wherein a first set of said dilation components are coupled to said first hinge tracks, a second set of said dilation components are coupled to said second hinge tracks, and a third set of dilation components are coupled to said third hinge tracks, and
wherein a first rotational movement applied to said first body component is converted to a first translational movement of said first set of dilation components, a second rotational movement applied to said third body component is converted to a second translational movement of said second set of dilation components, and a third rotational movement applied to said fourth body component is converted to a third translational movement of said third set of dilation components.
